SEQUENCE LISTING (4 sequences)

SEQ ID NO: 1 - DNA, 34 bp, Artificial
  Synthesized
gatgaagcttgccatggtggtattcaccatcctc

SEQ ID NO: 2 - DNA, 27 bp, Artificial
  Synthesized
tccgaattcaagcatgcacattggtag

SEQ ID NO: 3 - DNA, 26 bp, Artificial
  Synthesized
atgaagcttccaccatgtggctcgcg

SEQ ID NO: 4 - DNA, 27 bp, Artificial
  Synthesized
ctgcagaattcaagcatgcacattggt

(19) United States
(12) Patent Application Publication
(10) Pub. No.: US 2007/0190617 A1
(43) Pub. Date: Aug. 16, 2007

(54) DNA vaccine for Japanese encephalitis virus

(76) Inventors: Chang-Jer Wu, Taipei (TW); Mi-Hua Tao, Taipei (TW)

Correspondence Address: Frenkel & Associates, P.C., Suite 330, 3975 University Drive, Fairfax, VA, 22030, US

(21) Appl. No.: 11/353,212

(22) Filed: Feb. 13, 2006

(51) Int. Cl. C12Q 1/68; C12P 19/34

(52) U.S. Cl. 435/91.1; 435/6

(57) ABSTRACT

A DNA vaccine of Japanese encephalitis virus (JEV) of the present invention comprises at least one vector encoding a membrane protein and an envelope protein gene of JEV. Furthermore, the DNA vaccine contains a cytomegalovirus early promoter sequence, an enhancer sequence, a chimeric intron, a bovine growth hormone polyadenylation sequence and a kanamycin resistant gene. The DNA vaccine can enhance antigentic stability and provide a high level of immunity.

### BACKGROUND IF THE INVENTION

1. Field of the Invention

The present invention relates to a DNA vaccine for Japanese encephalitis virus, which is clinically used and highly efficient.

2. Description of Related Art

Japanese encephalitis virus (JEV) is a mosquito-transmitted, zoonotic flavivirus that affects a large portion of Asia occupied by some 40% of the world's population. Encephalitis caused by JEV has a high mortality and high case fatality rate. The use of a mouse brain-derived, formalin-fixed killed vaccine has brought the encephalitic case down to 10-30 cases per year as opposed to thousand of cases per year before the vaccine era. However, the in vivo production of this vaccine using many animals is becoming less acceptable against the background of newer technology. In addition, the adverse effects, such as allergic responses and neurotoxicity caused by the mouse brain-derived vaccine are also becoming less acceptable. Other major problems associated with the use of inactivated JEV vaccine are the relatively high cost of production and lack of long-term immunity. At least three doses of inactivated JEV vaccine are recommended to increase seroconversion rates, to raise antibody titers and to lengthen the duration of antibody persistence in vaccines.

Direct injection of the plasmid DNA in vivo results in the synthesis of viral proteins in the host and may mimic the action of attenuated vaccines. In fact, immunization with antigen encoding plasmid DNA has been demonstrated in animals ranging from mice to nonhuman primates to induce a broad range of immune responses, including humoral immune responses and cell-mediated immunity against pathogens, e.g., influenza and rabies viruses, malaria parasites and Mycobacterium tuberculosis.

Recently researches reported a range of protection rates, ranging from 28% to 100%, in contrast to the generally high responsiveness seen within mice. Efforts to optimize immune responses to DNA vaccine have included evaluation of DNA doses, immunization schedules, injection routes and delivery methods. Several factors could reflect the efficiency of expression of antigen genes and the immunogenicity of DNA vaccines, including the choice of the transcriptional elements used to drive antigen gene expression. However, little has been done to investigate the effect of regulator elements in the generation of immune responses to DNA vaccines. A systematic evaluation of the various sequence elements that contribute to high levels of expression in the target cells is the first step in developing an optimal vector.

Promoters differ in tissue specificity and efficiency in initiating mRNA synthesis. To date, most DNA vaccines in mammalian systems have relied upon viral promoters derived from cytomegalovirs (CMV). These CMV promoters have had good efficiency in both muscle and skin inoculation in a number of mammalian species.

The beneficial effect of introns on expression has been ascribed primarily to an enhanced rate of RNA polyadenylation and nuclear transport associated with RNA splicing but may also reflect the presence of transcriptional enhancers within the intron.

Transcriptional terminators are not widely recognized as gene regulatory elements. However, the efficiency of primary RNA transcript processing and polyadenylation are known to vary between transcriptional terminators of different genes.

The prokaryotic antibiotic resistance gene and backbone elements were altered and undesired viral sequences were removed to produce a plasmid vector more acceptable for future clinical use. The Amp gene is commonly employed as a selection marker for the production of plasmid DNA. However, penicillin and other lactam antibiotics can cause allergic reactions in certain individuals. According to “Points to Consider on Plasmid DNA for Preventive Infectious Disease Indications” released in 1996 by the Center for Biologies Evaluation and Research (CBER) of the Food and Drug Administration (FDA) in the USA, the Kan' gene is more appropriate to use in such a plasmid. This is mainly because kanamycin and other aminoglycoside antibiotics are not extensively used in the treatment of clinical infections.

In addition, proper structural conformation was altered to produce immune responses and protective immunity for DNA vaccines. Enveloped JEV particles contain a single, positive-polarity, 11-kb RNA genome. Three structural proteins, the capsid (C), the membrane (M), and the envelope (E), and seven non-structural proteins are all derived from a single long open reading frame. The non-glycosylated M protein is processed from a glycosylated precursor (prM). Co-synthesis of prM with the E protein is necessary for proper foldings membrane association and assembly of the latter protein. In previous studies, the plasmid pE used encodes the full-length E protein with only 15 amino acids from the C-terminal end of the M protein serving as a signal sequence. The pE-encoded E protein will likely adopt an improper structural conformation, which may explain the low Plaque Reduction Neutralization Test (PRNT) titer generated by this particular JEV DNA vaccine made of the pE plasmid.

Intramuscular immunization with DNA vaccines has been shown in many animal models to induce a broad range of immune responses and protective immunity. However, DNA vaccines are less effective in primates. One reason may be that the low expression of vectors is insufficient to trigger immune responses in primates.

To overcome the shortcomings, the present invention provides a highly efficient DNA vaccine for Japanese encephalitis virus to obviate or mitigate the aforementioned problems.

### SUMMARY OF THE INVENTION

The main objective of the invention is to provide a DNA vaccine for Japanese encephalitis virus that comprises at least a vector and can elicit high levels of immunity.

The DNA vaccine in accordance with the present invention for Japanese encephalitis virus (JEV) comprises at least one vector. The vector or vectors encode a membrane protein and an envelope protein of JEV and may further contain a polyadenylation sequence, an intron, a drug resistant gene, a promoter sequence and an enhancer sequence.

The present invention is further related to a vector for use as a DNA vaccine for JEV that comprises at cast a sequence encoding a membrane protein and an envelope protein of JEV.

The vector for use as a DNA vaccine for JEV may further contain a polyadenylation sequence, an intron, a drug resistant gene, a promoter sequence and an enhancer sequence.

Other objects, advantages and novel features of the invention will become more apparent from the following detailed description when taken in conjunction with the accompanying figures.

### DESCRIPTION OF THE DRAWINGS

FIG. 1 is a graph of luciferase activity of different vectors in vitro;

FIG. 2 is a Dot-blot assay of cells from various expressed JEV E protein vectors;

FIG. 3A is a graph of JEV DNA vaccine-induced protective immunity induced by various expressed JEV E protein vectors;

FIG. 3B is a graph of JEV lethality after injection of JEV DNA vaccines in FIG. 3A;

FIG. 4A is a graph of structural conformation on JEV DNA vaccine-induced protective immunity with and without cardiotoxin pretreatment;

FIG. 4B is a graph of JEV lethality after injection of JEV DNA vaccines in FIG. 4A;

FIG. 5 is a Western-blot assay of serum from mice injected with pE or pCJ-3/ME without cardiotoxin pretreatment;

FIG. 6A is an electron microscopy image of JEV-infected BHK cells; cells;

FIG. 6B is an electron microscopy image of pCJ-3/ME transfected BHK cells;

FIG. 7A is a graph of JEV DNA vaccine-induced protective immunity induced by various expressed JEV E protein vectors; and

FIG. 7B is a graph of the effectiveness of one dose on the JEV DNA vaccine in FIG. 7A.

### DETAILED DESCRIPTION OF PREFERRED EMBODIMENT

A DNA vaccine in accordance with the present invention for Japanese encephalitis virus (JEV) comprises at least one vector. The vector or vectors encode a membrane protein and an envelope protein of JEV and may further contain a polyadenylation sequence, an intron, a drug resistant gene, a promoter sequence and at least one enhancer sequence. Preferably, a single vector encodes the membrane protein and the envelope protein of JEV.

Preferably, the polyadenylation sequence is a bovine growth hormone polyadenylation sequence.

Preferably, the intron is a chimeric intron that contains a 5′-splice donor site from a first intron of a human β-globin gene and a 3′-splice acceptor site from the heavy chain variable region of an immunoglobulin gene.

Preferably, the drug resistant gene is a kanamycin resistant gene.

The promoter sequence drives the expression of the membrane protein and the envelope protein of JEV and is a cytomegalovirus early promoter.

A vector for use as a DNA vaccine in accordance with the present invention for JEV comprises at least a sequence encoding a membrane protein and an envelope protein of JEV and may further contain a polyadenylation sequence, an intron, a drug resistant gene, a promoter sequence and at least one enhancer sequence. Preferably, a single vector encodes the membrane protein and the envelope protein of JEV.

Preferably, the polyadenylation sequence of the vector for use as a DNA vaccine is a bovine growth hormone polyadenylation sequence.

Preferably, the intron of the vector for use as a DNA vaccine is a chimeric intron that contains a 5′-splice donor site from a first intron of a human β-globin gene and a 3′-splice acceptor site from an immunoglobulin gene heavy chain variable region.

Preferably, the drug resistant gene of the, vector for use as a DNA vaccine is a kanamycin resistant gene.

The promoter sequence of the vector for use as a DNA vaccine that drives the expression of the membrane protein and the envelope protein of JEV and is a cytomegalovirus early promoter.

As used in this description, definitions of the terms “DNA vaccine” and “vector” are provided to avoid any ambiguity in the description.

The term “DNA vaccine” refers to direct injection of a gene or genes coded for a specific antigenic protein or proteins, which result in direct production of a corresponding antigen or antigens in the vaccine recipient to trigger an appropriate immune response.

The term “vectors” refers to a DNA vector that can encode a membrane protein, an envelope protein of JEV or both. Furthermore, the vector may contain sequences including a polyadenylation sequence, an intron, a drug resistant gene, a promoter sequence and at least one enhancer sequence.

Two regulatory elements, a chimeric intron and a bovine growth hormone (BGH) polyadenylation sequence, proved to increase antigen expression in transfected cells following DNA vaccination, and a plasmid DNA encoding proteins of Japanese encephalitis virus demonstrated to be an effect DNA vaccine.

The DNA vaccine for Japanese encephalitis virus can be injected into mammals and is not limited in primates.

The JEV strain used in the following examples was Beijing-1 that was maintained in suckling mouse brains for preparation of a virus stock used for cloning of the JEV genes as well as for setting up a JEV challenge model. Female C3H/HeN mice were purchased from National Laboratory Animal Breeding and Research Center, Taipei, Taiwan. Mice were housed at the Laboratory Animal Facility, College of Life Sciences, National Taiwan Ocean University, Keelung, Taiwan. The 50% lethal dose (LD₅₀) of 12-14-week-old C3H/HeN mice against Beijing-1 JEV were calculated to be 3.0×10⁵ PFU. For a lethal challenge experiment, C3H/HeN mice were intraperitoneal inoculated with JEV Beijing-1 at a dose of 50 times the LD₅₀ followed by a sham intracerebral inoculation. The JEV-challenged mice were observed for symptoms of viral encephalitis and death every day for 30 days.

### EXAMPLES

The following examples are provided to illustrate various aspects of the present invention to a person knowledgeable in the art but do not limit the claims in any manner whatsoever.

### Example 1

### Construction of Expression Vectors

An eukaryotic expression vector refers to a vector capable of expressing sequences in an eukaryotic expression system and contains at least one promoter sequence and may further contain at least one enhancer sequence. The eukaryotic expression vector pcDNA (Invitrogen, San Diego, Calif., USA) contains a cytomegalovirus early promoter and enhancer sequence, a bovine growth hormone polyadenylation sequence, an ampicillin resistant gene and a neomycin selective arker gene. The vector p3224 contains a cytomegalovirus early promoter and enhancer sequence, intron A of the cytomegalovirus, a bovine growth hormone polyadenylation sequence and a kanamycin resistant gene. A BglII/BamI-II fragment of pRL-CMV (Promega, Madison, Wis., USA) and an XbaI/SalI fragment of p3224 were ligated to construct a chimeric vector pCJ-1 containing a Renilla luciferase gene. The Renilla luciferase gene of the vector pCJ-1 was removed and was designated as pCJ-2. The pCJ-2 contained only one NotI cloning site, so a multiple cloning site was inserted into the pCJ-2 vector to construct pCJ-2′. This vector contained the cytomegalovirus early promoter and enhancer sequence, the chimeric intron, the SV40 polyadenylation and the kanamycin resistant gene. The SV40 polyadenylation sequence of the pCJ-2′ was replaced by the bovine growth hormone polyadenylation sequence and was designated as pCJ-3. The pCJ-3 vector contained the cytomegalovirus early promoter, the enhancer sequence, the chimeric intron, the BGH polyadenylation and the kanamycin resistant gene.

The expressive level of various construct vectors was checked by a luciferase reporter system.

The firefly luciferase gene (from pGL3-basic; Promega, Madison, Wis., USA) was constructed for the construct vectors of pcDNA3, p3224, pCJ-2′ and pCJ-3. The resultant plasmids were designated respectively as pcDNA3/Luc, p3224/Luc, pCJ-2′/Luc and pCJ-3/Luc.

cDNA of the JEV envelope gene was obtained by reverse transcription and PCR amplification of the genomic RNA derived from the Beijing-1 JEV. The PCR reaction of a JEV E gene was performed using the primer set 5′-GATGAAGCTTGCCATGGTGGTATTCACCATCCTC-3′positive sense) (SEQ ID NO: 1) and 5′-TCCGAATTCAAGCATGCACATTGGTAG-3′ (negative sense) (SEQ ID NO: 2) to obtain a genetic fragment encoding the entire envelope protein (E) as well as a 15-amino-acid signal peptide derived from the C-terminus of the membrane protein (M). The PCR reaction of a JEV ME gene was performed using the primer set 5′-ATGAAGCTTCCACCATGTGGCTCGCG-3′ (positive sense) (SEQ ID NO: 3) and 5′-CTGCAGAATTCAAGCATOCACATTGGT-3′ (negative sense) (SEQ ID NO: 4) to obtain a genetic fragment encoding the entire membrane-envelope protein (ME) as well as a 15-amino-acid signal peptide derived from the C-terminus of the core protein (C).

A polylinker refers to a sequence comprising at least one restriction cleavage site. The fragment of the E and ME gene was cloned into the polylinker HindIII/EcoRI sites of plasmid pcDNA to produce the plasmid pE and pME,

The blunt-end fragment of the E gene was cloned into Klenow-fragment treated plasmids of pcDNA3, p3224, pCJ-2′ and pCJ-3. The resultant plasmids were designated as pE, p3224/E, pCJ-2′/E and pCJ-3/E.

The blunt-end fragment of the ME gene was cloned into Klenow-fragment treated plasmids of pcDNA3, p3224, pCJ-2′ and pCJ-3. The resultant plasmids were designated as pME, p3224/ME, pCJ-2′/ME, and pCJ-3/ME.

Plasmid DNA was purified from transformed Escherichia coli DH5α by Qiagen Plasmid Giga Kits (Qiagen, Hilden, Germany) in accordance with the manufacturer's instructions and stored at −70° C. as pellets. The DNA was reconstituted in sterile saline at a concentration of 1 mg/ml for experimental use.

### Example 2

### Effect of Different Introns and Poly(A) on Luciferase Expression

Previous studies in cultured cells and in transgenic mice have shown that the inclusion of heterologous introns in expression plasmids enhanced expression. The effect of two introns on expression was examined in vitro. One of the two introns contains the CMV intron A and a 3′-splice acceptor site from an immunoglobulin gene heavy chain variable region, while the other contains a 5′-splice donor site from a first intron of a human β-globin gene, and a 3′-splice acceptor site from an immunoglobulin gene heavy chain variable region.

To determine if the poly(A) signal sequence affected the expression, two different poly(A) signals, with one derived from the SV40 late gene and the other from the BGH gene, were compared. SV40 and BGH poly(A) signals are commonly used in expression vectors. The poly(A) signals were inserted into a vector containing the CMV promoter, luciferase reporter gene and the chimeric intron. The expression of various construct vectors was determined by the luciferase reporter system.

2.1 Cell Transfection

The BHK cell line was derived from the kidneys of 1-day-old hamsters and was used as an experiment platform for viral infection against mammal cells. About 2×10⁵ BHK cells (ATCC CCL-10) were seeded into each well in a 6-well plate and replaced with fresh medium before four hours of transfection. With the calcium phosphate precipitation method, 5 μg of DNA was first dissolved in 220 μl of 0.1×TE (1 mM Tris-Cl; 0.1 mM EDTA [pH 8.0]) buffer, and 250 μl of 2×HBS buffer (280 mM NaCl; 10 mM KCl; 1.5 mM Na2HPO4; 50 mM HEPES; 12 mM dextrose [pH 7.05]) was added to the solution. 31 μl of 2 M CaCl2 was then added and vortexed immediately. The solution stood for 25 minutes at room temperature. The transfection solution was added to the cell culture medium drop by drop and incubated at 37° C. for hours. The transfected medium was removed, and 15% of glycerol in 3×HBS was added for glycerol shock for 2 minutes at room temperature. The 15% glycerol in 1×HBS was removed and washed twice with 1×PBS. Fresh medium was then added to each well and incubated at 370° C.

2.2 Luciferase Assay

After the transfection, cells were washed twice with 1×PBS and treated with 200 μl of 1× reporter lysis buffer (RLB) (Promega, Madison, Wis., USA). The cell lysates were briefly centrifuged to pellet large debris, and the protein-concentration of the supernatant was measured by the BCA protein assay reagent (Pierce, USA). The 20 μl cell lysates (25 μg of total protein) were reacted with 100 μl of luciferase assay reagent (Promega, Madison, Wis., USA) at room temperature in a luminometer. The activity of plasmid expression was determined as a relative luciferase activity unit (RLU) per 25 μg of the total protein extract.

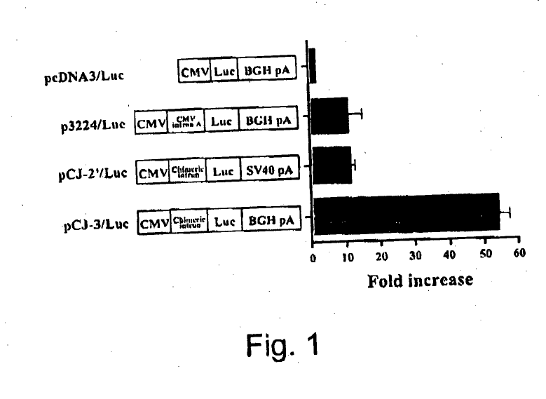

With reference to FIG. 1, the luciferase assay exhibited the expression levels of various construct vectors in BHK cells. The data were the average of six independent transfection experiments in duplicates. The pCJ-3/Luc was shown to be a highly expressive vector with an expression of 1.6×10⁷ relative luciferase activity units (RLU) in BHK cell. The p3224/Luc expressed 3.4×10⁶ RLU, whereas the pcDNA3/Luc only expressed 3.0×10⁵ RLU. The increase of pcDNA3/Luc was used as a base for comparison. About a 53-fold increase was observed in BHK cells expressing pCJ-3/Luc over the pcDNA3/Luc, whereas the p3224/Luc had an 11-fold increase over the pcDNA3/Luc.

The vector pCJ-3 containing the BGH poly(A) expressed 1.6×10⁷ RLU and was approximately 5 times higher than that observed with the SV40 poly(A) signal with an expression of 3.6×10⁶ RLU (pCJ-2′).

2.3 Dot-Blot Analysis

The transiently transfected cells were washed twice with cold PBS and lysed by addition of 300 μl of NP-40 lysis buffer (1% NP-40, 150 mM NaCl, 50 mM Tris [pH 8.01], protease inhibitor cocktail [Boehringer Mannheim, Mannheim, Germany]). Following centrifugation at 10,000×g for 10 min at 4° C., the lysates were analyzed by sodium dodecyl sulfate-polyacrylamide gel electrophoresis (SDS-PAGE [12.5% polyacrylamide]) and transferred to nitrocellulose membrane in a transfer buffer (0.1% SDS, 25 mM Tris [pH 8.4], 192 mM glycine, 20% methanol) for 2 hours at 50 mA. The filters were first treated with blocking buffer (5% skim milk, 150 mM NaCl, 50 mM Tris [pH 8.01]) for 2 hours and then incubated with JEV anti-E MAb E3.3 (1 μg/ml) for 1 hour at room temperature. Following four 5-min washings in washing buffer (0.05% Tween 20, 1% skim milk, 150 mM Tris [pH 8.01]), the membranes were incubated for 1 hour with goat anti-mouse lgG Fc-horseradish peroxidase (HRIP) (1:1000; Chemicon, Temecula, Calif., USA) in PBS-bovine serum albumin (1%). After six 5-min washings, the blots were developed by an enhanced chemiluminescence Western blot detection system (Amersham, Little Chalfont, UK) and exposed to X-ray film.

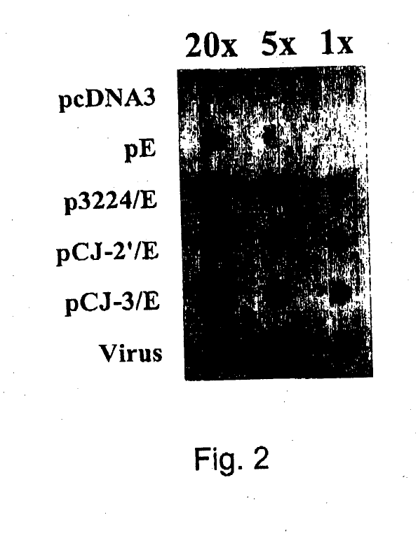

The expressive levels of these plasmids in BHK cells were confirmed by dot-blot assay. With reference to FIG. 2, the expression level of pCJ-3/E had a 3-fold increase over the p3224/E. About a 10-fold increase over the pE was observed in BHK cells expressing p3224/E. The above results thus indicated that the introns of the various construct vectors are critical for the expression. The chimeric intron is found to be the superior intron to CMV Intron A.

The relative levels of JEV E protein between two different poly(A) signals were also measured directly using Dot-blot assay. A 3-fold increase in the expression of JEV E protein in the BGH poly(A) signal vector was observed in comparison to the SV40 poly(A) signal vector.

### Example 3

### Effect of Different Intron and Poly(A) on the Antibody Response and Protection

To verify which plasmid could provide highly protective immunity against JEV infection, female C3H/HeN mice were immunized by intramuscular injection with each plasmid encoding JEV E-protein and followed by lethal JEV challenge. C3H/HeN mice were initially chosen in this challenge study because they were reported to be more sensitive to JEV infection than other inbred mouse strains.

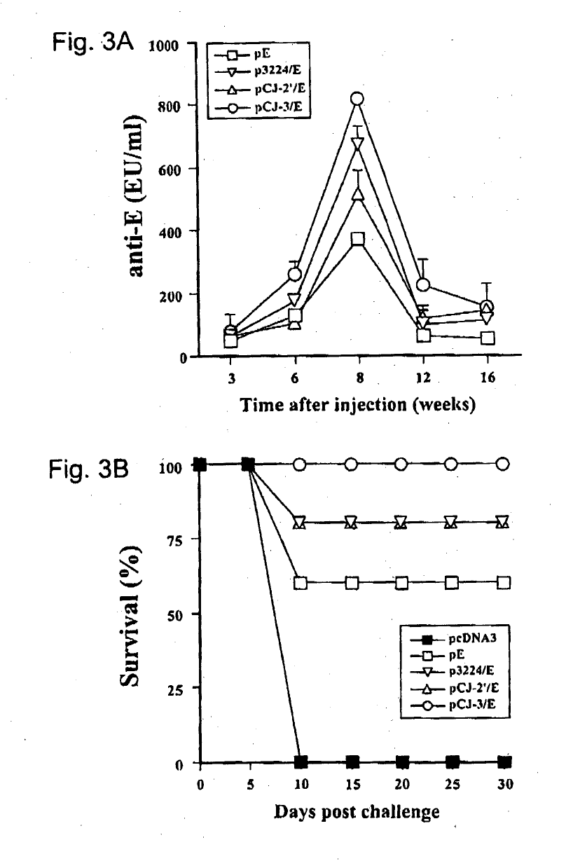

For intramuscular DNA immunization, all mice were immunized at to 8 weeks of age. In brief, groups of live mice were anesthetized and injected at 3-week intervals with 50 μg of DNA bilaterally into each quadricep muscle pretreated 1 week earlier with 100 μl of 10 μM cardiotoxin (Sigma, St. Louis, Mo., USA). With reference to FIG. 3A, serum samples were collected by tail bleeding at different times and analyzed for the presence of JEV E-specific antibodies, and results were recorded. Antibodies to JEV E were measured by ELISA. End-point titers were defined as the highest serum dilution that resulted in an absorbance value two times greater than that of non-immune serum with a cutoff dilution of 0.05 times. Samples below the limit of detection were assigned a value of 10, since the tested serum was diluted starting from a dilution of 1:10.

The results of JEV E-specific antibody analyses demonstrated that no significant difference exists in the production of JEV E-specific antibody at week 3. At week 6, the pCJ-3/E generated a 3-, 2-, 3-fold increase of the antibody respectively over the pCJ-2′/E, p3224/E, and pE, whereas with reference to FIG. 3A, pCJ-3/E generated a 1.6-, 1-, 2-fold increases at week 8 respectively over the pCJ-2′/E, p3224/E, and pE. The survival rates of the virus challenges at 16 weeks after the single immunization demonstrated that the pCJ-3/E provided full protection against the virus challenge, whereas with reference to FIG. 3B, the p3224/E and the pCJ-2′/E both resulted in a 80% survival rate, and the pE, a 60% survival rate.

Taken together, these data clearly indicate that a chimeric intron and the bovine growth hormone polyadenylation sequence increased the efficiency of muscle-targeted gene expression in vitro and immunities in vivo.

### Example 4

### Effect of Structural Conformation on the Antibody Response and Protection

4.1 Effect of Structural Conformation on JEV DNA Vaccine-Induced Protective Immunity

Cardiotoxin or bupivacaine pretreatment, which induces muscle necrosis and regeneration, is believed to enhance the efficiency of DNA plasmid uptake and expression in muscles; but JEV DNA vaccine given intramuscularly was previously found to be less effective without cardiotoxin pretreatment. To determine whether the proper structural conformation enhanced the efficacy of JEV DNA vaccine in the absence of cardiotoxin pretreatment, cardiotoxin-pretreated or control C3H/HeN mice were injected in the quadricep muscles with 100 μg of pE, pME or pCJ-3/ME DNA.

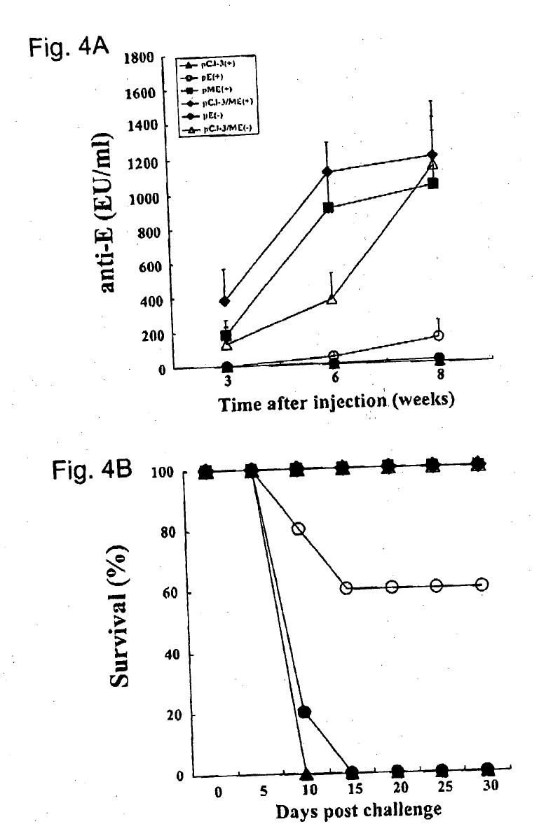

With reference to FIG. 4, the plasmids encoding the E proteins alone administered without cardiotoxin pretreatment similarly did not produce the antibody and induce protection. With reference to FIG. 4A, the results at week 6 demonstrated that the pCJ-3/ME with cardiotoxin pretreatment generated a 3-fold increase of the antibody over pCJ-3/ME without cardiotoxin pretreatment, whereas at week 8, pCJ-3/ME with or without cardiotoxin pretreatment generated no significant difference in the production of JEV E-specific antibody. At week 6 when pretreated with cardiotoxin, the pCJ-3/ME generated 25-fold and 1.3-fold increases of the antibody respectively over the pE and pME, whereas with reference to FIG. 4A, pCJ-3/ME generated a 9-fold and 1-fold increase at week 8 respectively over the pE and pME. The survival rates of the virus challenges at 8 weeks after the first immunization demonstrated that pCJ-ME with or without cardiotoxin pretreatment and pME with cardiotoxin pretreatment provided full protection against the virus challenge, whereas with reference to FIG. 4B, pE with cardiotoxin pretreatment demonstrated a 60% survival rate.

These results show that JEV DNA vaccine encoding prM and E given by intramuscular injection induces higher antibody titers and confers protection, with or without cardiotoxin pretreatment, against lethal JEV challenge.

4.2 Western-Blot Assay of Serum

To determine whether the serum has specific anti-JEV E antibody in the mice without cardiotoxin pretreatment, plasmid pE or pCJ-3/ME was administered by intramuscular injection into C3H/HeN mice without cardiotoxin pretreatment at 3-weeks intervals, and the serum at different times was measured by Western-blot assay.

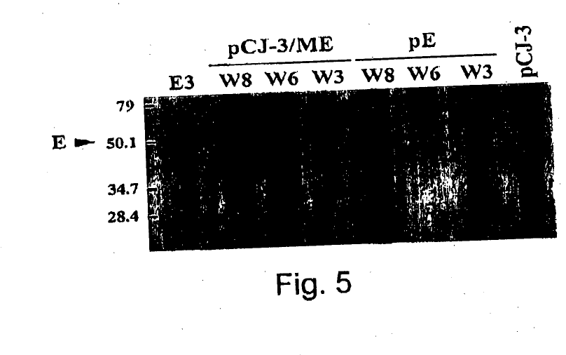

With reference to FIG. 5, the serum from pCJ-3/ME could clearly detect the 52 KDs of JEV E protein, but the serum from pE could not. A time-dependent increase of anti-JEV E antibody was evident.

4.3 Electron Microsopy

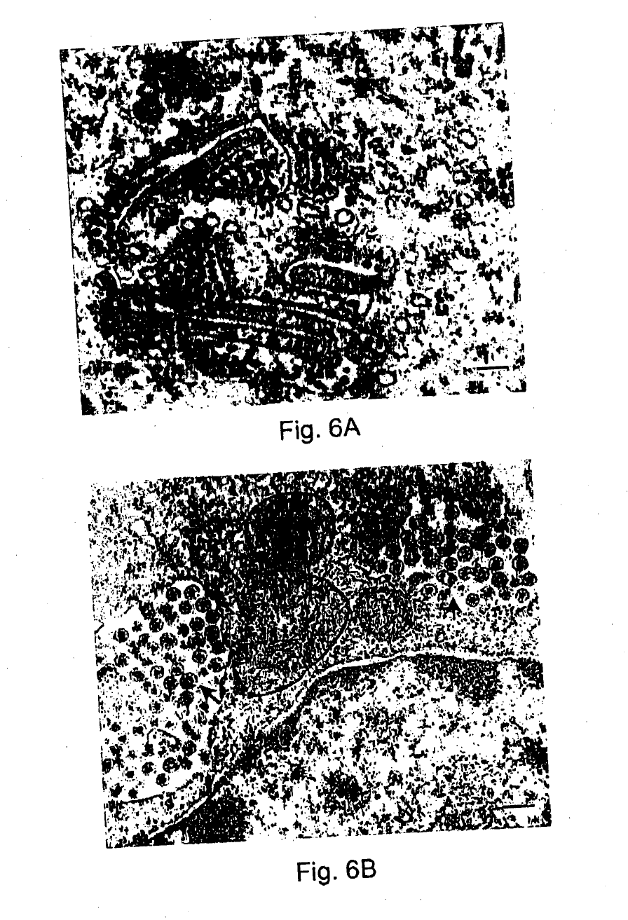

With reference to FIG. 6, to determine whether the proper structural conformation was altered to produce immune responses and protective immunity for DNA vaccine, the morphology of pCJ-3/ME transfected BHK-21 cells was detected by transmission electron microscopy.

For electron microscopy, the above cells were collected as cell pellets at 48 hours post infection, fixed in 2.5% glutaraldehyde containing 0.1 M cacodylate buffer (pH 7.4) for 60 min and washed overnight in the same buffer. The cell pellets were then stained and blocked with uranyl acetate, postfixed with osmium, dehydrated with graded ethanol and embedded ill Eponate-12 resin. Thin sections were double stained with uranyl acetate and lead citrate and examined under a Zeiss 900 electron microscope (Carl Zeiss, Germany).

With reference to FIG. 6A and B the appearance of pCJ-3/ME transfected BHK-21 cells was a normal type similar to that of parent cells. Many small particle-like and electron-dense structures were present in the cisternae of the ER and within the Golgi apparatus of pCJ-3/ME transfected BHK-21 cells, whereas such structures were not observed in an uninfected BHK-21 cell. These results suggest that the JEV DNA vaccine that expresses the prM and E proteins of JEV, which produced virus-like particles of JEV in vitro, was an effective DNA vaccine without cardiotoxin in a murine model.

### Example 5

### Effects of DNA Vaccine Immunized Once on the Antibody Response and Protection

5.1 Effect on One Dose JEV DNA Vaccine-Induced Protective Immunity

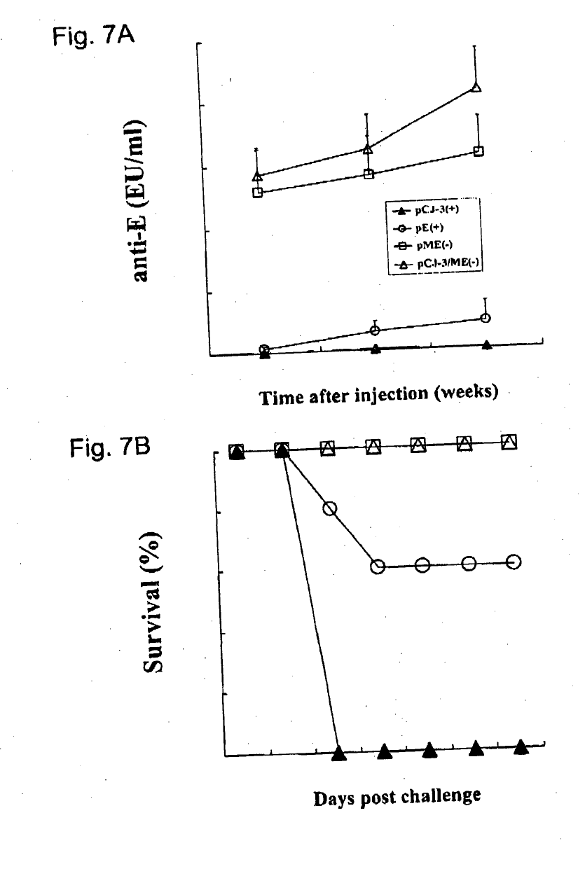

To determine whether one immunization had a similar effect, plasmid pE was administered by intramuscular injection into groups of cardiotoxin pretreated C3H/HeN mice. Animals receiving one dose of pCJ-3 served as negative controls. All mice were challenged 8 weeks after the first immunization with 50 LD₅₀ (3×10⁷ PFU) of JEV Beijing-1, and with reference to FIG. 7, the results were recorded. With reference to FIG. 7A, at week 3 and 6, the pME or pCJ-3/ME without cardiotoxin pretreatment generated no significant difference in the production of JEV E-specific antibody, whereas at week 8, pCJ-3/ME generated a 1.3-fold increase of the antibody over pME. However, at week 3, 6 and 8, the pCJ-3/ME, without cardiotoxin pretreatment generated 44-fold, 10.6-22 fold and 9.4-fold increases of the JEV E-specific antibody over pE with cardiotoxin pretreatment. As expected, none of the mice in the control pCJ-3 groups survived the JEV challenge.

With reference to FIG. 7B, mice that received pME and pCJ-3/ME by intramuscular immunization one time without cardiotoxin pretreatment were all significantly protected in that 100% of the mice survived the challenge (>30 days after viral challenge).

5.2 Induction of Neutralizing Antibodies in Mice

TABLE 1
Induction of neutralizing antibodies in
mice immunized with JEVDNA vaccines

| Immunized | Neutralizing antibody^{b} | | | | |
|---|---|---|---|---|---|
| dose^{a} | pCJ-3(+) | pE(−) | pE(+) | pCJ-3/ME(−) | pCJ-3/ME(+) |
| 1 | <1/10 | <1/10 | 1/10 | 1/160 | 1/160 |
| 3 | <1/10 | <1/10 | 1/10 | >1/320 | >1/320 |

^{a}Groups of 6-8 week-old C3H/HeN mice were intramuscular immunized once or three times at 3-week intervals of pCJ-3, pE and pCJ-3/ME with (+) or without (−) cardiotoxin pretrratment.
^{b}JEV-neutralizing titers in serum collected at 8 weeks after the first immunization were expressed as the reciprocal of the serum dilution yielding a 50% reduction in plaque number.

The ability of the antiserum of different immunized groups to neutralize JEV infection in vitro was carried out by Plaque Reduction Neutralization Tests (PRNT). Groups of 6-8 week-old C3H/HeN mice were immunized intramuscularly once or three times at 3-week intervals with pCJ-3, pE and pCJ-3/ME with (+) or without (−) cardiotoxin pretreatment. At 8 weeks after the first immunization, sera were collected and checked for JEV-neutralizing antibodies. PRNT titers were not detectable (<1:10) in mice immunized with the pE DNA vaccine delivered by intramuscular with cardiotoxin pretreatment or none (Table 1). Mice immunized with the pCJ-3/ME DNA vaccine had significantly increased PRNT titers (1:320) in their prechallenge sera. These results demonstrated that one dose of pCJ-3/ME immunization was more effective in JEV-specific neutralizing JEV antibody and protective levels in mice without cardiotoxin pretreatment than the pE administered mice pretreated with cardiotoxin.

### CONCLUSION

A good DNA vaccine input have a stronger expressive system and a proper structural conformation of antigen. The present invention demonstrates that a plasmid DNA encoding prM and E, which produces virus-like particles of JEV in vitro, is an effective DNA vaccine without a cardiotoxin. The plasmid DNA encoding prM and E enhances antigenic stability and provides a high-density presentation to antigen-presenting cells. The DNA vaccine comprises the plasmid encoding prM and E elicits high levels of immunities and is high efficient.

Various modifications and variations of the present invention will be recognized by those persons skilled in the art without departing from the scope and spirit of the invention. Although the invention has been described in connection with specific preferred embodiments, the invention as claimed should not be unduly limited to such specific embodiments. Indeed, various modifications of the described modes for carrying out the invention, which are obvious to those skilled in the art, are intended to be within the scope of the following claims.

What is claimed is:

1. A DNA vaccine for Japanese encephalitis virus (JEV) comprising at least one vector, wherein the vector or vectors encode a membrane protein and an envelope protein of JEV.

2. The DNA vaccine as claimed in claim 1 comprising a single vector encoding a membrane protein and an envelope protein of JEV.

3. The DNA vaccine as claimed in claim 2, wherein the vector further contains a polyadenylation sequence.

4. The DNA vaccine of JEV as claimed in claim 2, wherein the vector further contains an intron.

5. The DNA vaccine as claimed in claim 2, wherein the vector further contains a drug resistant gene.

6. The DNA vaccine as claimed in claim 2, wherein the vector further contains a promoter sequence.

7. The DNA vaccine of JEV as claimed in claim 3, wherein the polyadenylation sequence is a bovine growth hormone polyadenylation sequence.

8. The DNA vaccine as claimed in claim 4, wherein the intron is a chimeric intron that contains a 5′-splice donor site from a first intron of a human β-globin gene and a 3′-splice acceptor site from an immunoglobulin gene heavy chain variable region.

9. The DNA vaccine as claimed in claim 5, wherein the drug resistant gene is a kanamycin resistant gene.

10. The DNA vaccine as claimed in claim 9, wherein the promoter sequence drives the expression of the membrane protein and the envelope protein of JEV and is a cytomegalovirus early promoter.

11. The DNA vaccine of JEV as claimed in claim 2, wherein the vector further contains at least one enhancer sequence.

12. A vector for use as a DNA vaccine comprising at least a sequence encoding a membrane protein and an envelope protein of JEV.

13. The vector as claimed in claim 12, which further contains a polyadenylation sequence.

14. The vector as claimed in claim 12, which further contains an intron.

15. The vector as claimed in claim 12, which further contains a drug resistant gene.

16. The vector as claimed in claim 12, which further contains a promoter sequence.

17. The vector as claimed in claim 13, wherein the polyadenylation sequence is a bovine growth hormone polyadenylation sequence.

18. The vector as claimed in claim 14, wherein the intron is a chimeric intron that contains a 5′-splice donor site from a first intron of a human β-globin gene and a 3′-splice acceptor site from an immunoglobulin gene heavy chain variable region.

19. The vector as claimed in claim 15, wherein the drug resistant gene is a kanamycin resistant gene.

20. The vector as claimed in claim 19, wherein the promoter sequence drives the expression of the membrane protein and the envelope protein of JEV and is a cytomegalovirus early promoter.

21. The vector as claimed in claim 12, which further contains at least one enhancer sequence.
